(19) United States
(12) Patent Application Publication
(10) Pub. No.: US 2009/0306568 A1
(43) Pub. Date: Dec. 10, 2009

(54) INTERMITTENT LUMBAR TRACTION APPARATUS AND METHOD

(76) Inventor: DONALD W. MEYER, Fountain Valley, CA (US)

Correspondence Address: STETINA BRUNDA GARRED & BRUCKER, 75 ENTERPRISE, SUITE 250, ALISO VIEJO, CA, 92656, US

(21) Appl. No.: 12/135,763

(22) Filed: Jun. 9, 2008

(51) Int. Cl. A61F 5/00

(52) U.S. Cl. 602/33; 602/32; 602/36; 602/4

(57) ABSTRACT

A traction method and apparatus for treating irregularities in the lateral curvature and global posture of the thoraco-lumbar, lumbar, lumbo-pelvic and/or pelvic region of the spine involving the application of an intermittent transverse traction load to a region of the patient's body. The patient is placed in a supine or semi-supine position, and a traction sling is positioned about a region of the body of the patient, where the region of the body can be the thoracic region, lumbar region and pelvic region of the body. A transverse traction load having an intermittently varying magnitude is exerted on the sling to induce an extension posture in at least a portion of the spine of the patient and/or a flexion posture of the pelvic portion of the spine of the patient. The flexion and extension postures serve to at least partially restore normal lateral curvature of the thoraco-lumbo-pelvic region of the spine, thereby reducing lower back pain, correcting abnormal posture, and easing other symptoms that are related to thoraco-lumbo-pelvic spine curvature irregularities.

### CROSS-REFERENCE TO RELATED APPLICATIONS

Not Applicable.

### STATEMENT RE: FEDERALLY SPONSORED RESEARCH/DEVELOPMENT

Not Applicable

### BACKGROUND OF THE INVENTION

1. Technical Field

The present invention relates generally to an apparatus and method for achieving traction of at least one of the thoraco-lumbar, lumbo-pelvic and pelvic region of the spine to treat conditions related to irregular lateral curvature of the thoraco-lumbo-pelvic region, including lumbogenic pain symptoms of mechanical origin related to reduced lumbar lordosis/extension and altered posture, such as abnormal anterior or posterior thoracic translation with associated increased or decreased lateral sacral inclination.

2. Related Art

Proper anterior to posterior alignment and lateral curvature of the spine are known to be important to maintain good posture and function of the spine. FIG. 1A-1C illustrate peer reviewed and published ideal lateral curvature of a spinal column 8, with FIGS. 1A and IC showing the semi-circular curvature through the cervical region (C1-C7) 10, the elliptical curvature of the thoracic region (T1-T12) 13, and the lumbar region (L1-L5) 15. FIG. 1B illustrates the same spinal column 8 as viewed from the back of the individual, and showing the different regions of the spine 8. As show in FIGS. 1A and 1C, the thoracic region 13 of the spinal column 8 has an elliptical concave curvature as viewed from the front of the individual. In contrast the cervical region 10 (located in the region of the neck) has a semi-circular convex curvature, which convexity is echoed in the elliptical curvature of the lumbar region 15 (located in the region of the lower back). Improper anterior to posterior alignment and/or lateral curvature of regions of the spinal column 8 can develop through accident or trauma, as well as through illness such as tumor or osteoporosis (bone density loss), and also through idiopathic causes or in the normal process of aging, as in spondylosis (also known as degenerative joint disease.) Such irregular lateral curvature and alignment and posture of the spine 8 is often accompanied by reduced motion, pain and discomfort, which may severely reduce and impair the quality of life, and can even be disabling for such individuals. For example, improper lateral curvature of the lumbar region 15 of the spine 8 may take the form of hypolordosis or kyphosis, which is a partial or complete loss or reversal of the natural curvature of the lumbar region 15 (i.e. lumbar lordosis) that can cause lower back pain and discomfort. In fact, a reduced lumbar lordosis with or without associated anterior or posterior thoracic translation and reduced or increased lateral sacral inclination is one of the most cited physical features associated with chronic low back pain in subjects in the current research literature.

One method that has been used for the treatment of lower back pain involves lumbar traction, in which at least a portion of the lumbar region of the spinal column is stretched or extended to achieve axial distraction of the spine and reduce axial stresses on the discs. Such lumbar traction can be achieved through the use of, for example, backswings that allow a patient to hang upside down by their feet, or via shoulder harnesses that vertically suspend the upper body of the patient, allowing the spinal column to extend under the weight of the patient. However, while such methods may be suitable for reducing lordosis, i.e. excessive curvature of the spine, they may not be suitable for patients having hypolordosis, or a loss in the curvature of the spine.

In a more recent method, traction of the lumbar region of the spine is achieved by strapping a patient in on an examination/treatment table, fitting a portion of the patient's torso with a traction strap, and exerting a static traction load to pull the strap and thereby transversely stretch the spine. In these methods, the static traction force applied to the body induces an extension posture in the lumbar region of the spine that is maintained at an intensity and for a duration sufficient to induce musculoligamentous changes, thereby “remodeling”the spine into a more proper lateral curvature and posture. This method is thus effective to increase and shape the curvature of the spine to combat conditions such as hypolordosis or kyphosis. Further description of this and other lumbar traction methods are described in more detail in the Harrison CBP Seminars, Inc. Publication, Chapter 5, the “History of Lumbar Traction,”having a copyright date of 2004, which is herein incorporated by reference in its entirety.

However, a problem with such conventional static, extension traction methods is that they often do not achieve proper remodeling of the spine within an acceptable time frame. For example, because the traction force applied during treatment is limited by the patient's tolerance level for the magnitude of the force being applied, proper treatment of hypolordosis of the lumbar region of the spine may require an excessive number of repeated lumbar traction sessions in order to achieve the desired treatment. This can be a problem both because the scheduling and coordinating of multiple sessions can be inconvenient for many patients, and also patient compliance with treatment programs is often decreased when numerous sessions are involved. Patients may also continue to suffer excessive levels of lower back pain while waiting to complete the required traction sessions. Yet another problem with conventional extension traction methods is that most patients experience discomfort during prolonged static traction sessions, due to constant unchanging tension exerted on the spine. Also, the effectiveness of the traction treatment may decrease over time as a result of the contracture or activation of muscles in response to the traction force (i.e., “muscle-guarding” ). Accordingly, there remains a need for a safe and efficient means of treating patients having an abnormal or irregular lateral curvature and posture of the spine, without excessive pain or discomfort.

### BRIEF SUMMARY OF THE INVENTION

The present invention specifically addresses and alleviates the above-identified deficiencies in the art. In this regard, the present invention is directed to a method of treating irregularities in one or more of the lateral curvature and posture of at least one of the thoraco-lumbar, lumbar, lumbo-pelvic, and pelvic regions of the spine in a patient by performing a traction method that incorporates a transverse traction load having an intermittently varying magnitude. A patient is placed in a supine or semi-supine position, and a traction sling is positioned about a region of the body of the patient, where the region of the body can be the thoracic region, lumbar region and pelvic region of the body. A transverse traction load is exerted on the sling to induce an extension posture in at least a portion of the spine of the patient, the transverse traction load having a magnitude that is intermittently varying. The extension posture serves to at least partially restore curvature of the region of the spine, such as the lumbar region, thereby increasing range of motion and reducing symptoms such as lower back pain, abnormal posture, and other symptoms of mechanical origin that are related to spinal curvature irregularities.

In one version, the intermittent traction load has a non-zero magnitude that varies sinusoidally. For example, the intermittent traction load can vary sinusoidally from a maximum to a minimum magnitude having a difference of about 1 lb to about 30 lbs. A frequency of variation of the intermittent traction load may be from about 5 cycles/min to about 20 cycles/min. The traction sling can be positioned and the transverse traction load can be applied at angles selected to provide the desired lateral curvature of the spine, as well as the desired lateral inclination of the sacrum.

A traction apparatus can be provided to perform the intermittent lumbar traction, the apparatus having a traction sling that is sized and configured to fit about the region of the body of the patient, and a traction motor capable of exerting the intermittent traction load. A load transfer line is provided that is in mechanical communication with the traction sling and traction motor, and that transfers the traction load from the traction motor to the traction sling. A traction support anchors the load transfer line such that the traction sling is pulled in a direction having a transverse component with respect to a longitudinal axis of the supine or semi-supine patient upon exertion of the traction load, such as in an upward direction. The exertion of the traction load induces an extension posture in at least a portion of the spine of the patient, thereby effecting the traction of the spine.

In one version, the traction motor has an off-state and an on-state, with the intermittent traction load being exerted while the traction motor is in the on-state. In yet another version, the traction support includes a base portion having parallel base beams and an upper portion comprising parallel upwardly extending beams and a horizontal top bar connecting the upwardly extending beams, the horizontal top bar having at least one pulley mounted thereon to anchor the load transfer line to the traction support at a position above the patient.

The present invention is best understood by reference to the following detailed description when read in conjunction with the accompanying drawings.

### BRIEF DESCRIPTION OF THE DRAWINGS

These as well as other features of the present invention will become more apparent upon reference to the drawings wherein:

FIG. 1A is a schematic side view of a spinal column having normal curvature and showing the cervical, thoracic and lumbar regions of the spine;

FIG. 1B is a schematic back view of the spinal column of FIG. 1A;

FIG. 1C is a schematic side view showing the curvature of the spinal column of FIG. 1A;

FIG. 2 is schematic front view of an embodiment of a traction apparatus comprising a traction motor capable of outputting an intermittent traction load according to the instant invention;

FIG. 2A is a schematic side view of an embodiment of a rope ratchet mechanism and rope ratchet guide plate suitable for use with the traction apparatus of FIG. 2;

FIGS. 3A-3B are schematic side views of embodiments of traction apparatus capable of effecting traction in the spine of a patient, according to the instant invention;

FIG. 4A is a schematic front view of a version of a traction motor capable of outputting a traction load having an intermittently varying magnitude;

FIG. 4B is schematic sectional front view of the traction motor of FIG. 4A;

FIGS. 5A-5G are schematic side views of embodiments of lumbar traction apparatus having configurations for effecting supine lumbar traction according to the present invention.

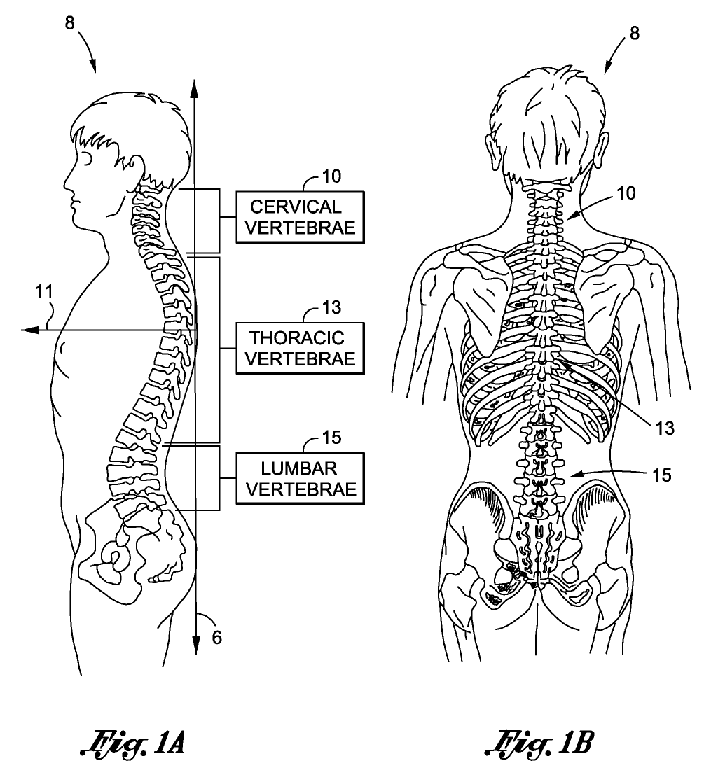

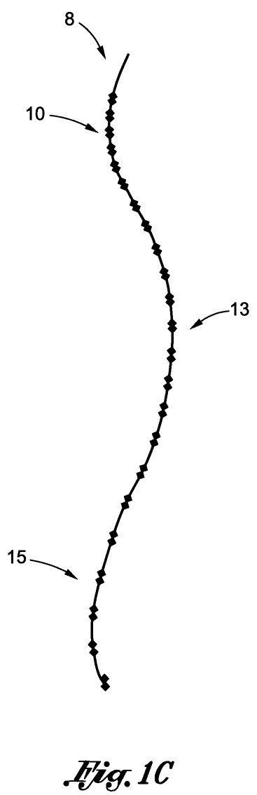

Common reference numerals are used throughout the drawings and detailed description to indicate like elements.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

It has been discovered that the application of an intermittent traction load can be used to enhance traction of the lumbar region 15 of the spine 8. The application of the intermittent traction load increases invertebral separation and patient comfort thereby increasing the effectiveness of the traction. This should reduce the duration and frequency of lumbar traction sessions necessary to restore a more normal thoraco-lumbo-pelvic spinal structure/posture. By “intermittent traction load”it is meant that the traction load (i.e. traction force) that is applied to achieve lumbar traction has a magnitude that varies over a selected duration, such as from a minimum traction load to a maximum traction load, as opposed to a “static”traction load that is applied at a substantially continuous and unchanging magnitude. Without being bound by any theory, it is believed that the application of the intermittent traction load decreases the “muscle-guarding”response that is otherwise elicited in static traction methods, which improves intervertebral separation and patient comfort during the traction process which thereby increases the effectiveness of the treatment.

The intermittent traction load can be applied at magnitudes and frequencies that are selected according to factors such as the type and severity of the spinal condition being treated, as well as the age, body size and tolerance level of the patient and the number and duration of traction sessions to be administered. The intermittent traction load is a non-zero load applied over a preselected period of time, and having a varying, non-zero magnitude. In one version, the intermittent traction load has a continuously varying magnitude that cycles from a minimum traction load level to a maximum traction load level at a pre-selected frequency. Such a continuously varying traction load could also be characterized as a having sinusoidally varying magnitude, and may be desirable to reduce “jarring”of the patient that might other wise occur with sudden or abrupt changes in traction load. As an example, the intermittent traction load may substantially continuously vary in magnitude from a minimum traction load to a maximum traction load having a difference in load poundage of from about 1 to about 30 lbs, such as from about 10 to about 20 pounds or from about 15 to about 25 pounds. The frequency of the traction load variation cycle may be from about 5 cycles/min to about 20 cycles/min, such as about 12 cycles/min. In one version, the rate of resting human respiration, approximately 12-13 cycled/min, is utilized as the traction frequency to improve patient relaxation. Alternatively, the frequency of the variation in the traction load and/or the minimum and maximum traction loads may be increased or decreased through the duration of the lumbar traction session, or increased or decreased between sessions. For example, a maximum traction load applied in an initial traction session may be from about 15 to about 35 lbs, such as from about 20 to about 30 lbs, which maximum traction load can be increased with subsequent sessions but typically should not be allowed to exceed more than one-half of the patient's body weight.

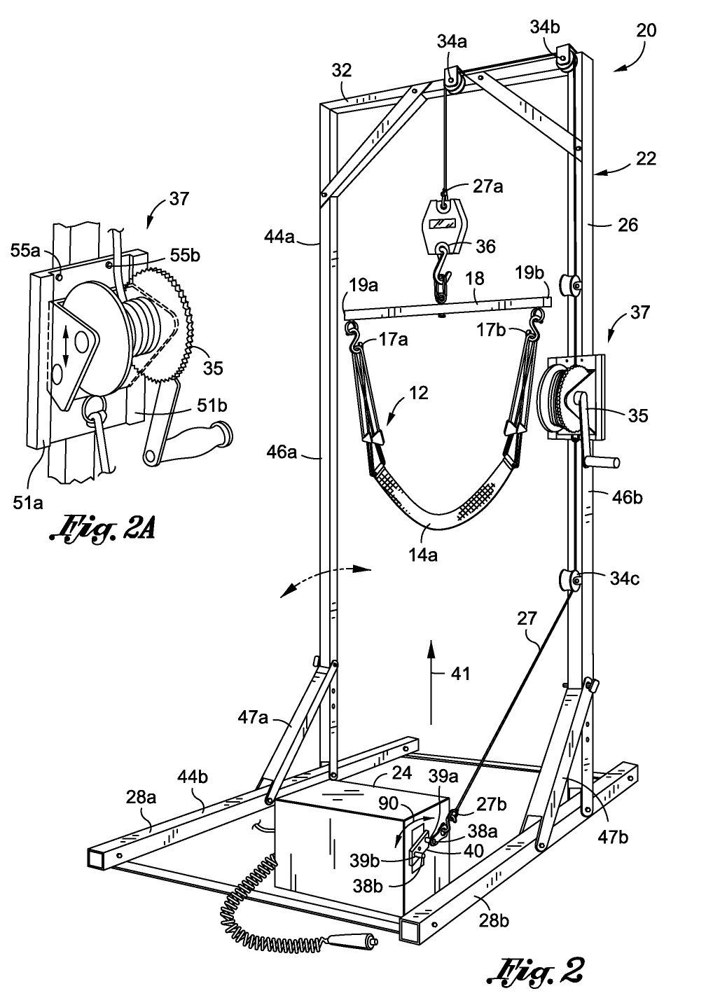

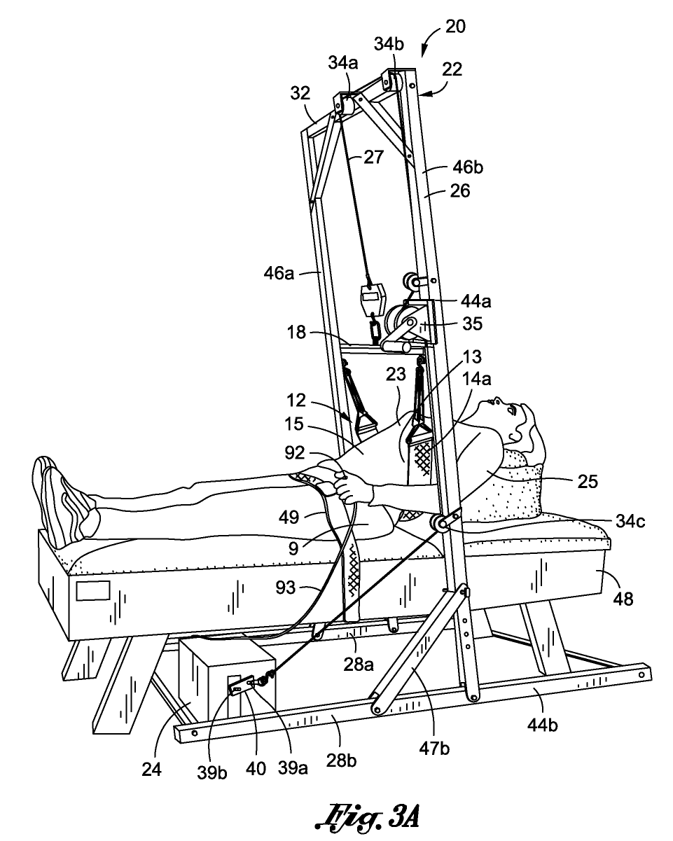

FIG. 2 shows an example of a traction apparatus 20 suitable for use with the application of an intermittent traction load according to the instant invention. The traction apparatus 20 generally comprises a traction sling 12, traction motor 24, traction load transfer line 27, and a traction support 22. The traction sling 12 is capable of being fitted about a region 23 of the body of the patient 25, as shown for example in FIGS. 3A and 3B, such as a region 23 of the body selected from the group consisting of the thoracic region 13, the lumbar region 15 and the pelvic region 9 of the patient's body. For example, the traction sling 12 can comprise one or more traction straps 14a, 14b, which can be padded for the patient's comfort, and which are sized, shaped and configured to at least partially encircle the region 23 of the patient's body. The traction straps 14a, 14b have sufficient strength to allow the region 23 of the body to be at least partially lifted when a traction load is applied to pull on the traction sling 12, preferably without excessive discomfort to the patient. In the version shown in FIG. 2 the traction apparatus 20 further comprises a horizontal spreader bar 18 with first and second ends 19a, 19b, that are connected to first and second ends 17a, 17b of the traction sling 12. The horizontal spreader bar 18 enhances the efficiency and comfort of the application of the traction load to the patient 25, by providing a more uniform application of the load to the patient's body region 23, thereby reducing squeezing or pinching of the patient by the straps 14a, 14b, as well as by facilitating easy entry and exit of the patient into and out of the traction sling 12.

The traction apparatus 20 further comprises a load transfer line 27 that has a first end 27a that is in mechanical communication with the traction sling 12, and a second end 27b that is in mechanical communication with the traction motor 24, and that is capable of transferring a traction load output from the traction motor 24 to the traction sling 12. For example, the load transfer line 27 may comprise one or more ropes, wires, and similar elements that are linked together to mechanically connect the motor 24 to the traction sling 12, the load transfer line 27 being capable of being disposed in tension with the traction sling 12 and traction motor 24. A transfer of the traction load from the motor 24 to the traction sling 12 can proceed by exertion of a traction load or traction force that pulls on the transfer line 27, the force of which pull is mechanically transmitted through the line 27 and to the traction sling 12. The transfer of this traction load pull to the traction sling 12 thus results in a substantially simultaneous pull and/or lift on the region 23 of the patient's body about which the traction sling 12 is fitted. Conversely, a decrease in the traction load output by the motor 24 at least partially releases the load transfer line 27, and lessens the pull on the traction sling 12 and the region 23 of body the patient 25. The load transfer line 27 may further comprise other elements in the line that aid in application of the traction load. For example, as shown in FIG. 2, the load transfer line 27 can comprise a scale 36 such as an in-line spring scale (not shown) or a hanging digital tension scale (shown) that allows measurement of the traction load being applied. Also, the hanging digital tension scale may serve to protect the patient by having a tension “failure limit”beyond which the scale will initiate and audible alarm, thereby protecting the patient from excessive traction loads. In the version shown in FIG. 2, the load transfer line 27 includes a digital scale 36 having a bottom hook that is attached to the horizontal spreader bar 18 from which the traction sling 12 is suspended.

The traction apparatus 20 further comprises a traction motor 24 that is capable of exerting the traction load having the magnitude that is intermittently varying, as described above. In one version, the traction motor 24 is capable of exerting the intermittent traction load by pulling on the line 27 connecting the motor 24 to the traction sling 12 with a force having a magnitude that is varied according to the desired intermittent traction load application. For example, the motor 24 may exert an intermittent force that alternately increases and decreases the pull on the line 27, thereby increasing or decreasing the tension in the line 27 and magnitude of the traction load being applied to the patient's body region 23. In one version, the traction motor 24 has an on-state in which the traction load is exerted at the intermittently varying magnitudes. The traction motor 24 may also have an off-state where either no traction load is exerted, or the traction load being exerted on the patient's body region is static and non-varying, depending upon the configuration of the apparatus 20.

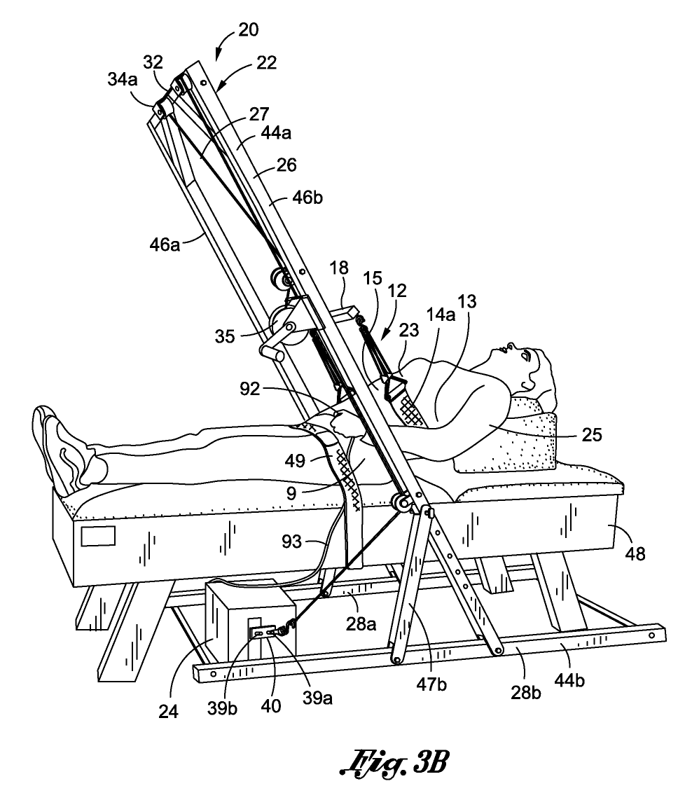

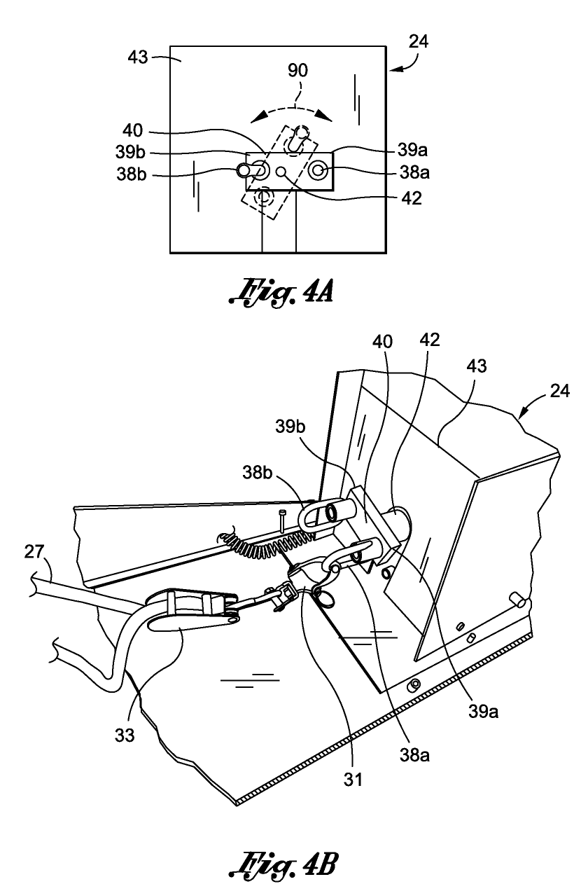

An example of a traction motor 24 capable of providing such an intermittent traction force is a rotating motor or eccentric motor, as show for example in FIGS. 4A-4B. In this version, the rotating motor 24 comprises one or more hook-ups 38a, 38b, to which the line 27 can be connected, which are disposed towards the distal ends 39a, 39b of a rotatable rod 40. The rotatable rod 40 is attached to a pin 42 that extends from within the motor casing 43, and that rotates upon activation of the motor 24. Activation of the rotating motor 24, for example by powering up via an external or internal power source (not shown), thus results in rotation of the pin 42 and simultaneous rotation of the rod 40 in a circular path 90 that is in a plane defined by the length of the rod 40, such as in a vertical plane in the version shown in FIG. 4A. The rotating movement of the rod 40 pulls the line 27 with a force that varies according to the position of the rod 40 in the circular path. For example, the force exerted on the line 27 is the least when the rod end 39a to which the line 27 is connected is rotated in a direction that decreases the tension in the line 27, and the force exerted is greatest when the rod end 39a is rotated in a direction that increases tension in the line. For example, in the version shown in FIGS. 3A and 3B, a lighter traction load is applied when the end 39a of the rod 40 is rotated in a direction that is towards a direction of travel of the line 27 away from the motor 24, and the load continuously increases throughout the rotation of the rod 40 until the heaviest load is achieved at the point where the rod end 39a is rotated furthest away from the direction of travel of the line 27 away from the motor 24, such as at a point that is 180 degrees from the direction of travel of the line 27 away from the motor 24 and towards the traction support 22. Thus, by rotating the rod 40 in a circular motion, the traction force applied to the traction sling 12 and patient 25 can be intermittently decreased and increased in magnitude in a sinusoidal fashion. In yet another aspect, the rod 40 may be laterally off-set with respect to the position of the pin 42, and thus may comprise a more lateral hook-up 38a and a more medial hook-up 39b, either of which can be selected for connection to the line 27. The more lateral hook-up carves out a circular path having a greater diameter, resulting in a greater difference between the maximum and minimum traction load magnitudes, whereas the more medial hook-up 39b carves out a smaller circular path, resulting in a smaller magnitude difference. Similarly, it can be understood that the length of the rod 40 can be selected according to the traction load differential that is desired. To inhibit the line 27 from twisting or knotting during the rotating operation of the motor 24, the motor can comprise hook-ups 38a, 38b that are swiveling hook-ups, capable of rotating independently of the rod 40. The line 27 can also be attached to the hook ups 38a, 39b via one or more snap swivels 31 that have a swiveling base that can rotate 180° independently of a clip portion that attaches to the hook-ups 38a, 38b, as shown for example in FIG. 4B. As is also shown in FIG. 4B, the line 27 can be connected to the snap swivel 31 via a rope ratchet 33, which can assist in maintaining the desired tension in the line 27 and can also be used to increase and/or decrease the tension in the line 27.

The traction apparatus 20 further comprises a traction support 22 that is capable of supporting the load transfer line 27. The traction support 22 may be configured to support the load transfer line 27 such that the traction sling 12 is pulled in a direction having a transverse component with respect to a longitudinal axis 16 of the spine 8 of the supine or semi-supine patient 25 upon exertion of the traction load. By having a transverse component it is meant that the traction load is applied in a direction having a vector component that is normal to a longitudinal axis 16 of the patient's spine, although the actual direction of the pull (the sum of the vector components) may be either parallel or at an angle to such normal. In other words, the traction support 22 is configured to allow the traction load applied to the traction sling 12 to exert an outwardly directed pull, such as a forward and/or upward pull, on the selected region 23 of the patient's body. A traction load applied to exert such a pull on the patient's body region 23 can also be referred to as a transverse traction load. The application of this transverse traction load induces an extension posture in at least a portion of the spine 8 of the patient 25, such as an outwardly directed extension of the spine 8 as shown in FIGS. 3A and 3B, that extends and separates segments of the spine 8, and which extension posture can at least partially restore the proper curvature of the spine 8, particularly in the lumbar region 15 of the spine 8. Without being bound by any theory, it is believed that the inducement of this extension posture may result in musculoligamentous changes in the region of the spine that can at least partially “remodel” the spine, and provide improved posture, improved structural curvature, and pain relief.

In the versions shown in FIGS. 2, 3A and 3B, the traction support 22 comprises an upright frame 26 that is configured to allow the traction sling 12 to be pulled at various different angles from above the patient 25, thereby achieving different types and levels of transverse traction. In this version, the traction frame 26 comprises an upper portion 44a pivotally engaged to a lower portion 44b. The upper portion 44a of the traction frame 26 comprises upwardly extending parallel beams 46a, 46b and a horizontal top cross bar 32 that connects the upwardly extending parallel beams 46a at the top of the beams 46a. The lower portion 44b of the traction frame 26 comprises parallel floor beams 28a, 28b that can be configured to counterbalance the weight of the upper portion 44a. The upwardly extending beams 46a, 46b can be pivotally attached to the parallel floor beams 28a, 28b, to allow angling of the upper portion 44a of the frame with respect to the lower portion 44b, and thus alteration of the angle of pull of the traction sling 12. For example, the upper portion 44a may be angled at from about 0° to about ±30°, such as ±45° with respect to a normal to the base portion 44b. In the version shown in FIG. 3a, the upper portion 44a is angled vertically at about 0° with respect to a normal 41 to the base portion 44b, which corresponds to a 90° angle with respect to a longitudinal axis 16 of the spine 8 of the supine or semi-supine patient 25. In the version shown in FIG. 3B, the upper portion 44a is angled at about 30° with respect to a normal to the base portion 44b, which is also about a 30° angle with respect to a normal 11 to the longitudinal axis 16 of the spine 8 of the patient 25. In one version, support beams 47a, 47b are slideably attached to and extend upwardly from the parallel floor beams 28a, 28b, and can be secured to the upwardly extending parallel beams 46a, 46b at a desired angle. Thus, the upper portion 44a of the frame 26 can be fixed at a desired angle with respect to the lower portion 44b by pivoting the upwardly extending parallel beams 46a, 46b to a desired angle, and securing the upper ends of the support beams 47a, 47b to the upwardly extending parallel beams 46a, 46b at a position along the length of the upwardly extending beams 46a, 46b that preserves the selected angle. The support beams 47a, 47b can be secured at the selected position via insertion of a cotter pin or other similar device.

The traction support 22 can also be configured to anchor the load transfer line 27 thereto at a position above the patient 25, thereby allowing for the application of a traction load having a transverse component. For example, in the version shown in FIG. 2, the traction support 22 comprises a pulley 34a mounted at about the middle of the horizontal top cross bar 32 through which the load transfer line 27 is threaded, to anchor the load transfer line 27 to the top cross bar 32. The traction sling 12 is suspended from one end 27a of the load transfer line 27 passing through the pulley 34a, thereby hanging the traction sling 12 from traction frame 26. The other end 27b of the load transfer line 27 may be passed through one or more subsequent pulleys 34b, 34c that are mounted on the support frame 26 before the end is connected to the traction motor 24. The traction support 22 may comprise one or more further pulleys, hooks, D-rings, and similar parts that are capable of supporting line 27 from the frame 26. The traction support 22 further comprises a ratchet winch 35 through which the line 27 is threaded that can be used to increase or decrease tension in the line 27 during lumbar traction sessions. Referring to FIG. 2, it can be seen that the ratchet winch 35 is supported on the traction frame by a guide plate 37 comprising a track with securing sides 51a, 51b that hold the ratchet winch 35. The ratchet winch 35 allows the tension in the line 27 to be increased and/or decreased according to the tension that is desired, and is slideable on the guide plate track within the sides 51a, 51b of the guide plate 37. The guide plate 37 further comprises pins 55a, 55b positioned at the top of the guide plate 37 that keep the ratchet winch 35 from sliding too far up and/or off of the guide plate 37. Thus, in operation, the ratchet winch 35 can be allowed to slide along the guide plate 37 during winching and/or other set up of the line 27, with the pins 55a, 55b and securing track sides 51a, 51b keeping the ratchet winch 35 from sliding too far. The guide plate 37 also secures the ratchet winch 35 and line 27 to the frame 26 during rotation of the upper portion of the frame 26.

In operation, the patient 25 is positioned in a supine or semi-supine position within the support frame 26, such as on an examination/treatment table 48 or examination chair, as shown for example in FIGS. 3A and 3B. The traction sling 12 is positioned about the desired region 23 of the patient's body, such as by wrapping the traction straps 14a, 14b under or behind the patient 25 at the desired region 23. The patient 25 may also be strapped down in one or more second regions 49 of the body, which may serve to oppose and intensify the applied traction force and increase or decrease pelvic flexion during the traction session. Also, supporting cushions or blocks can optionally be provided to position portions of the patient's body according to the desired traction posture. The load transfer line 27 is connected to the traction sling 12 and the traction motor 24, and the tension in the traction sling 12 can be incrementally increased or decreased, such as via the ratchet winch 35, until a desired initial transverse traction load is achieved. The traction motor 24 is typically maintained in the off-state while the patient is being loaded into the traction apparatus 20. The traction motor 24 can then be switched to the on-state to initiate the intermittent traction load application, such as by sinusoidally increasing and decreasing the pull on the transfer load line 27. The tension of the line 27 can also be further adjusted after activation of the motor 24 via the ratchet winch 35 or an in-line rope ratchet mechanism, such as the rope ratchet 33 shown in FIG. 4B. As a safety feature, the patient 25 can also be provided with a remote switch 92 that is in electrical communication with the motor 24, such as via an electrical cord 93, and that can be used to switch the motor 24 to the off-state according to the patient's needs. The application of the transverse traction load selectively pulls on the region 23 of the body about which the traction sling 12 is fitted, thereby inducing an extension in the spine 8 of the patient 25. In particular, the transverse traction load can be used to stretch the anterior spinal tissue, thereby extending the low back.

The duration and frequency of the traction sessions can be selected according to the type and severity of the condition being treated, as well as patient tolerance for the traction load. In one version, the intermittent traction load is applied for a session of from about 3 to 5 minutes, and the session is subsequently repeated for increasing durations, such as up to 15-20 minutes per session, with a minimum recommended traction session time of ten minutes and a maximum recommended traction session time of twenty minutes. It is know, from published ligamentous creep charts, that ligamentous deformation drastically reduces after approximately twenty minutes. A recommended frequency of the treatments may be at least two to three times per week. In initial sessions, the recommended maximum traction poundage may be from about 15 to about 35 lbs, such as from about 20 to about 30 lbs, which may be increased in subsequent sessions until either the patient's tolerance level or a maximum recommended amount of about one half the patient's body weight is reached. A differential between the maximum traction poundage and minimum traction poundage may be from about 1 to about 30 pounds, such as from about 10 to about 20 lbs. The lumbar traction method according to the instant invention can be performed to provide conservative treatment for painful lumbogenic conditions, including lower back pain, disc problems, hypolordosis, and other posture problems related to irregular curvature of the spine, by stretching the anterior spinal tissue, reducing the pressure on discs, as well as by effecting restoration of lumbar lordosis/pelvic tilt and reduction of excessive anterior head carriage or posterior rib carriage translation.

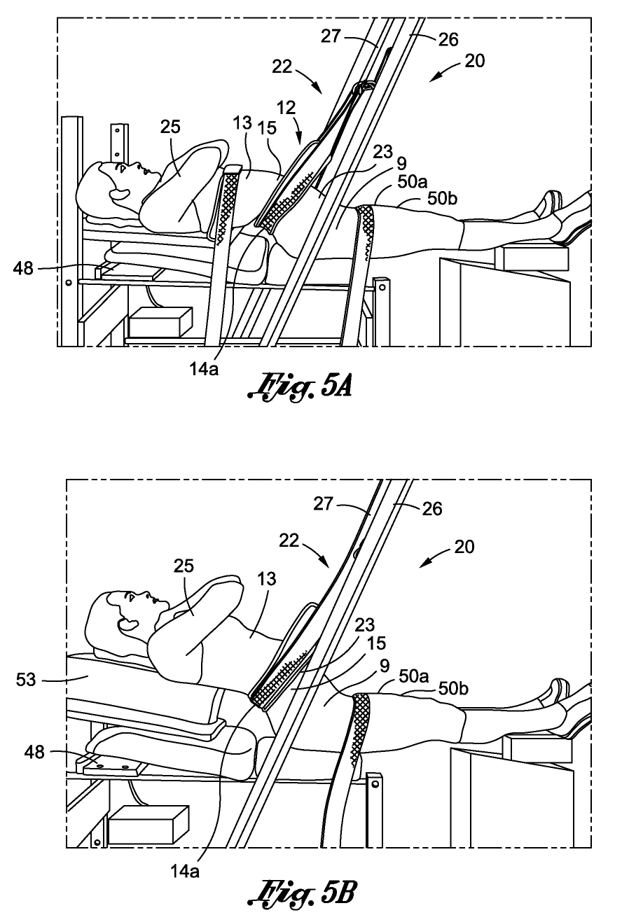

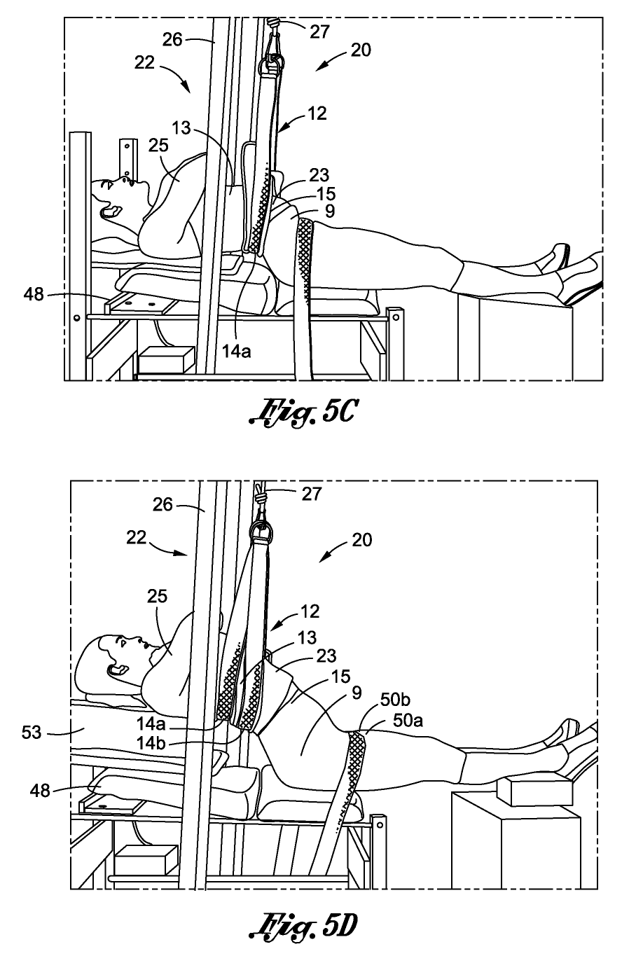

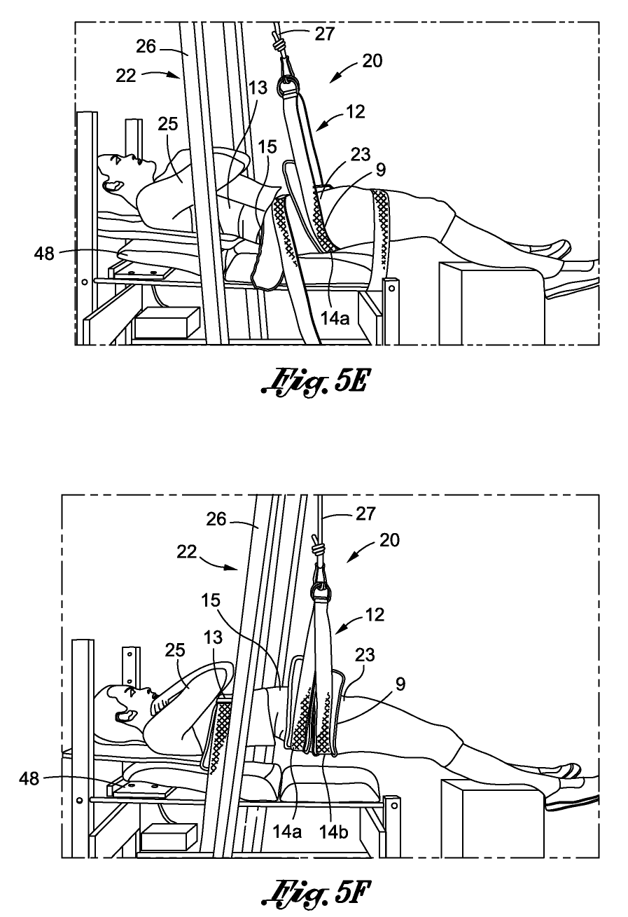

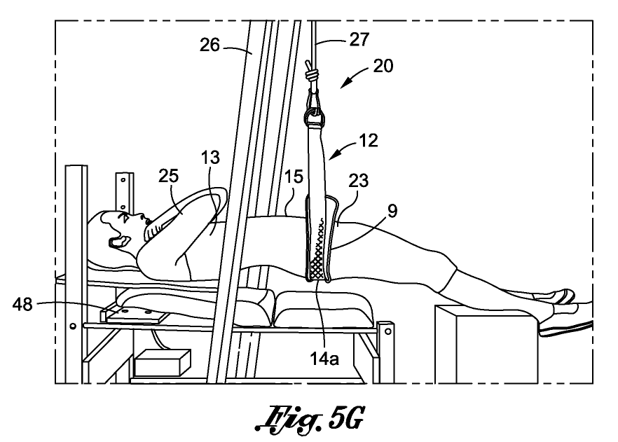

Methods of effecting lumbar traction with the traction apparatus 20 having the intermittent traction load application will be explained in more detail with regards to FIGS. 5A through 5G, which give examples of embodiments of spinal traction methods according to the instant invention. In each of the examples as shown below, it is recommended that the intermittent traction load be applied for about 1 to 3 minutes to start, with the duration of application of the intermittent traction load being increased another 1 to 3 minutes per session until 1 to 20 minutes of traction can be performed. Also, the tension in the load transfer line 27 can be increased with each traction session according to patient tolerance.

In the version shown in FIG. 5A, a supine lumbar traction method is performed by positioning the patient 25 in a supine position (laying down with face up), such as on an examination and/or treatment bench 48. The mid to lower rib cage of the patient 25 is strapped to the bench 48. The upper thighs (femurs) 50a, 50b of the patient 25 can also be strapped to the examination/treatment bench 48 to constrain them and allow the pelvis to flex upon the fixed hip joints. The traction sling 12 is fitted about the lower lumbar region 15 of the patient's body. The traction support 22 is configured such that the traction sling 12 is pulled at an angle that is offset from the vertical by about 10 degrees to 30 degrees, in the direction of the patient's feet. An intermittent force such as that described above is applied to output intermittent levels of traction load to the lower lumbar region 15 of the spine 8, thereby inducing an extension posture in the spine 8. This type of traction can increase distal lordosis of the lumbar region 15 of the spine 8, as well as increase the pelvic tilt and cause posterior thoracic translation, thereby remodeling and improving the curvature and posture of the spine 8.

In the version shown in FIG. 5B, a supine lumbar traction method is performed by positioning the patient 25 in a supine position, such as on an examination/treatment bench 48. The upper thighs (femurs) 50a, 50b of the patient 25 can be strapped to the examination/treatment bench 48 to constrain the upper thighs and allow the pelvis to flex upon the fixed hip joints. A 4-6 inch supporting block 53 is also placed behind the patient's torso. The traction sling 12 is fitted about the lower lumbar region 15 of the spine. The traction support 22 is configured such that the traction sling 12 is pulled at an angle that is offset from the vertical by about 10 degrees to 30 degrees, in the direction of the patient's feet. An intermittent force such as that described above is applied to output intermittent levels of traction load to the lower lumbar region 15 of the spine 8, thereby inducing an extension posture in the spine 8. This type of traction can increase distal lordosis of the lumbar region 15 of the spine 8, as well as increase the pelvic tilt and cause anterior thoracic translation, thereby remodeling and improving the curvature and posture of the spine.

Yet another version of a supine lumbar traction method is shown in FIG. 5C. In this version, the anterior superior pelvic region 9 of the patient 25 is strapped to the examination/treatment bench 48. The thorax of the patient is maintained in a neutral position. The traction sling 12 is fitted about the mid to upper lumbar region 15 of the spine 8. The traction support 22 is configured such that the traction sling 12 is pulled at an angle of about 80° to 90° with respect to the longitudinal axis 16 of the supine patient's spine 8, thereby pulling the mid to upper lumbar portion 15 of the spine 8 in an upward direction (transverse direction). An intermittent traction load such as that described above is output to apply intermittent levels of traction load to the mid to upper lumbar region 15 of the spine 8, thereby inducing an extension posture in the spine 8. This type of traction can be performed in cases where it is desired to increase the lordosis of the mid to upper lumbar region 15 of the spine 8, substantially without flexing and increasing the pelvic tilt.

In a further version of supine lumbar traction method as shown in FIG. 5D, the upper thighs (femurs) 50a, 50b of the patient 25 are strapped to the examination/treatment bench 48. A 4-6 inch supporting block 53 is placed behind the patient's torso. The traction sling 12 is fitted about the lower thoracic region 13 of the spine 8, and the traction support 22 is configured such that the traction sling 12 is pulled at 80° to 90° with respect to the longitudinal axis 16 of the supine patient's spine 8, thereby pulling the lower thoracic region 13 of the spine 8 in an upward direction. The intermittent force such as that described above is output to apply intermittent levels of traction load to the mid to upper lumbar region of the spine, thereby inducing an extension posture in the spine. This type of traction can be used in instances where it is desired to translate the thorax anteriorly as well as increase the pelvic tilt and the lordosis of the mid to upper lumbar region of the spine.

In a yet another version of a supine lumbar traction method as shown in FIG. 5E, the upper anterior pelvic region 9 of the patient 25 is strapped to the examination/treatment bench 48. The traction sling 12 is fitted about the lower pelvic region 9 of the patient 25. The traction support 22 is configured such that the traction sling 12 is pulled at an angle (such as at about 10 degrees) that rocks the pelvis towards the posterior of the patient's body (pelvic extension). The patient's lower thighs can also be secured and/or strapped to the examination/treatment bench 48, as needed. An intermittent force such as that described above is output to apply intermittent levels of traction load to the pelvic region 9, thereby posteriorly angling the pelvis. This type of traction can be used to create a backward bending and extension of the pelvis, thereby restoring a more normal pelvic tilt and decreasing any excessive curvature of the lower lumbar region 15 of the spine 8.

Yet another version of a supine lumbar traction method is shown in FIG. 5F. In this version, the mid to lower rib cage of the patient 25 is strapped to the bench 48. The traction sling 12 is fitted about the pelvic region 9 of the patient 25. The traction support 22 is configured such that the traction sling 12 is pulled at a 90° with respect to the longitudinal axis 16 of the patient's spine 8, thereby lifting the pelvis of the patient upwardly. An intermittent force such as that described above is output to apply intermittent levels of traction load to the pelvic region. This type of traction can be used in instances where it is desired to create a posterior thoracic translation posture.

A final example of a supine lumbar traction method is shown in FIG. 5G. In this version, the thorax and thighs of the patient remain neutral. The traction sling 12 is fitted about the pelvic region 9 of the patient 25, and the traction support 22 is configured such that the traction sling 12 is pulled at a 90° with respect to the longitudinal axis 16 of the patient's spine 8, thereby lifting the pelvis of the patient upwardly. An intermittent force such as that described above is output to apply intermittent levels of traction load to the pelvic region 9. This type of traction can create a thoracic extension that at least partially restores a proper curvature of the spine.

Additional modifications and improvements of the present invention may also be apparent to those of ordinary skill in the art. Thus, the particular combination of components and steps described and illustrated herein is intended to represent only certain embodiments of the present invention, and is not intended to serve as limitations of alternative devices and methods within the spirit and scope of the invention. Along these lines, it should be understood that the traction support 22, traction sling 12, traction motor 24 and traction line 27 may take any of a variety of forms that are known or later developed in the art, and further contemplates that such existing or newly formed traction components, such as newly formed traction frames 26 and traction straps 14, should fall within the scope of the present invention. Also, it should be understood that the traction apparatus 20 can comprise other configurations, and can be used to perform traction methods other than those specifically described. Similarly, the method of intermittent lumbar traction can be performed with apparatus and configurations other than those specifically described.

What is claimed is:

1. A method of treating irregularities in at least one of a lateral curvature and posture of one or more of a thoraco-lumbar, lumbar, lumbo-pelvic and pelvic region of a spine in a patient in need thereof, the method comprising the steps of:
(a) placing the patient in a supine or semi-supine position;
(b) positioning a traction sling about a region of the body of the patient, the region of the body being selected from the group consisting of the thoracic region, lumbar region and pelvic region of the body; and
(c) exerting a transverse traction load on the sling to induce an extension posture in at least a portion of the spine of the patient, the transverse traction load having an intermittently varying magnitude,
wherein the extension posture at least partially restores at least one of proper lateral curvature and posture of the thoraco-lumbar, lumbar, lumbo-pelvic and/or pelvic region of the spine in the patient.

2. A method according to claim 1, wherein the intermittently varying magnitude varies substantially continuously.

3. A method according to claim 1, wherein the intermittently varying magnitude comprises a sinusoidally varying magnitude.

4. A method according to claim 1, wherein the intermittently varying magnitude comprises a maximum magnitude and a minimum magnitude, and wherein a difference between the maximum and minimum magnitudes of the transverse traction load is from about 1 lb to about 30 lbs.

5. A method according to claim 4, wherein the difference is from about 10 lbs to about 20 lbs.

6. A method according to claim 1, wherein the intermittently varying magnitude varies with a frequency of from about 5 cycles/min to about 20 cycles/min.

7. A method according to claim 1, wherein the intermittent traction load is a non-zero load.

8. A method according to claim 1, wherein a second region of the patient's body is fixed to one or more of an examination and/or treatment bench or chair, the second region of the body comprising at least one of the thorax, pelvic and thigh regions of the body.

9. A method according to claim 1, wherein the traction sling is positioned about the lower lumbar region of the patient, and the transverse traction load is applied at an angle of about 10 to 30 degrees with respect to a normal to the longitudinal axis of the supine patient.

10. A method according to claim 1, wherein the traction sling is positioned about the mid to upper lumbar region of the patient or the thorax of the patient, and the transverse traction load is applied at about a 80 to 90 degree angle with respect to the longitudinal axis of the patient.

11. A method according to claim 1 wherein the traction sling is positioned about the pelvic region of the patient, and the transverse traction load is applied at about a 90 degree angle with respect to the longitudinal axis of the patient.

12. A traction apparatus for effecting traction of a spine of a patient, the apparatus comprising:
(a) a traction sling that is sized and configured to fit about a region of the body of the patient, the region of the body being selected from the group consisting of the thoracic region, lumbar region and pelvic region of the body;
(b) a traction motor capable of exerting a traction load having a magnitude that is intermittently varying;
(c) a load transfer line capable of being placed in mechanical communication with the traction motor and the traction sling to transfer the traction load exerted by the traction motor to the traction sling; and
(d) a traction support capable of supporting the load transfer line such that the traction sling is pulled in a direction having a transverse component with respect to a longitudinal axis of the patient upon exertion of the traction load, wherein exertion of the traction load induces an extension posture in at least a portion of the spine of the patient, thereby effecting traction of the spine.

13. A traction apparatus according to claim 12 wherein the traction motor has an off-state and an on-state, and wherein the traction motor exerts the traction load having the magnitude that is intermittently varying when in the on-state.

14. A traction apparatus according to claim 12 wherein the traction motor is capable of exerting a traction load having an intermittently varying magnitude that varies substantially continuously.

15. A traction apparatus according to claim 13 wherein the traction motor is capable of exerting a traction load having an intermittently varying magnitude that comprises a sinusoidally varying magnitude.

16. A traction apparatus according to claim 15, wherein the traction motor is capable of exerting a traction load having an intermittently varying magnitude that comprises a maximum magnitude and a minimum magnitude, and wherein a difference between the maximum and minimum magnitudes of the traction load is from about 1 lb to about 30 lbs.

17. A traction apparatus according to claim 16, wherein the traction motor is capable of exerting a traction load having a difference between the minimum and maximum magnitude of from about 10 lbs to about 20 lbs.

18. A traction apparatus according to claim 17, wherein the traction motor is capable of exerting a traction load having an intermittently varying magnitude that varies with a frequency of from about 5 cycles/min to about 20 cycles/min.

19. A traction apparatus according to claim 12, wherein the traction support comprises a base portion comprising parallel floor beams and an upper portion comprising upwardly extending parallel beams and a horizontal top bar connecting the upwardly extending parallel beams, the horizontal top bar having at least one pulley mounted thereon to anchor the load transfer line to the traction support at a position above the patient.

20. A traction apparatus according to claim 19 wherein the upper portion of the traction support is pivotally engageable to the base portion such that the upper portion is capable of being angled at from about 0 degrees to ±45 degrees with respect to a normal to the base portion.
